(19) United States
(12) Patent Application Publication
(10) Pub. No.: US 2017/0106119 A1
(43) Pub. Date: Apr. 20, 2017

(54) HEMOSTATIC AND ANTIMICROBIAL BONE MATRIX

(71) Applicant: Warsaw Orthopedic, Inc., Warsaw, IN (US)

(72) Inventors: Robert P. Skinner, Jackson, NJ (US); Guobao Wei, Milltown, NJ (US)

(21) Appl. No.: 14/886,181

(22) Filed: Oct. 19, 2015

(51) Int. Cl. A61L 27/54; A61L 27/36

(52) U.S. Cl. CPC A61L 27/54; A61L 27/365; A61L 2430/02; A61L 2300/404; A61L 2300/418

(57) ABSTRACT

Implantable matrices are provided that include at least one therapeutic agent having hemostatic and antimicrobial activity. The implantable matrices are configured to be implanted into a hone defect. The implantable matrices aid in reducing operative and post-operative bleeding and inhibiting microbial growth. In some embodiments, the matrices provided include therapeutic agent(s) having hemostatic and antimicrobial activity that do not compromise the bioactivity of the matrix to induce or permit new tissue growth.

### BACKGROUND

Bone is a composite material that is composed of impure hydroxyapatite, collagen and a variety of non-collagenous proteins, as well as embedded and adherent cells. Due to disease, a congenital defect or an accident, a person may lose or be missing part or all of one or more bones or regions of cartilage in his or her body, and/or have improper growth or formation of bone and/or cartilage.

Mammalian bone tissue is known to contain one or more proteinaceous materials that are active during growth and natural bone healing. These materials can induce a developmental cascade of cellular events that result in bone formation. Typically, the developmental cascade of bone formation involves chemotaxis of mesenchymal cells, proliferation of progenitor cells, differentiation of cartilage, vascular invasion, bone formation, remodeling and marrow differentiation.

When bone is damaged, often bone grafting procedures are performed to repair the damaged bone especially in cases where the damage is complex, poses a significant risk to the patient, and/or fails to heal properly. Bone grafting is also used to help fusion between vertebrae, correct deformities, or provide structural support for fractures of the spine. In addition to fracture repair, bone grafting is also used to repair defects in bone caused by birth defects, traumatic injury, or surgery for bone cancer.

There are at least three ways in which a bone graft can help repair a defect. The first is called osteogenesis, the formation of new bone within the graft. The second is osteoinduction, process in which molecules contained within the graft (e.g., bone morphogenic proteins) convert the patient's cells into cells that are capable of forming bone. The third is osteoconduction, a physical effect by which a matrix often containing graft material acts as a scaffold on which bone and cells in the recipient are able to form new bone.

The source of bone for grafting can be obtained from bones in the patient's own body (e.g., hip, skull, ribs, etc.), called an autograft, or from bone taken from other people that is frozen and stored in tissue banks, called an allograft. The source of bone may also be derived from animals of a different species called a xenograft.

Some grafting procedures utilize a variety of natural and synthetic matrices with or instead of bone (e.g., collagen, silicone, acrylics, hydroxyapatite, calcium sulfate, ceramics, etc.). To place the matrix at the bone defect, the surgeon makes an incision in the skin over the bone defect and shapes the matrix to tit into the defect.

During implantation of a bone graft, often times in surgery, the implant site involves significant blood loss or hemorrhaging, resulting in the need for suctioning and possibly cauterization. in addition, there is always potential for, and there have been occurrences of, infections during surgery and the post-operative stage.

Growth factors (e.g., bone morphogenic protein-2) may also be present in the graft in order to spur the patient's body to begin the formation of new bone and/or cartilage. These growth factors act much like a catalyst, encouraging the necessary cells (including, but not limited to, mesenchymal stem cells, osteoblasts, and osteoclasts) to more rapidly migrate into the matrix, which is eventually resorbed via a cell-mediated process and newly formed bone is deposited at or near the bone defect. In this manner severe fractures may be healed, and vertebrae successfully fused.

It would be beneficial to provide a bone graft that is hemostatic and well as having antimicrobial activity without compromising its biological and mechanical activities required to achieve new bone growth and effective repair of the bone defect site. Thus, there is a need to develop new matrices that improve bone and/or cartilage repair, and that address the hemostatic and antimicrobial problems discussed above.

### SUMMARY

Implantable matrices are provided that aid in the reduction of operative and post-operative bleeding and also kill and/or inhibit microbial growth. By using therapeutic agent(s) having hemostatic and antimicrobial activity in association with the matrix, the problems of excessive bleeding and microbial infection during and/or after surgery are addressed.

In some embodiments, the matrices provided include therapeutic agent(s) having hemostatic and antimicrobial activity that do not compromise the bioactivity of the matrix to induce or permit new tissue growth, e.g., new bone growth.

In one embodiment, there is an implantable matrix comprising an effective amount of at least one therapeutic agent having hemostatic and antimicrobial activity disposed in a biodegradable polymer, wherein the implantable matrix is configured to be implanted into a bone defect and release the therapeutic agent.

In some embodiments, the at least one therapeutic agent comprises a hemostatic agent and an antimicrobial agent. In some embodiments the hemostatic agent is present in an amount up to 10 wt % and the antimicrobial agent is present in an amount up to 1 wt %, based on the total weight of the matrix.

In some embodiments, the hemostatic agent comprises silver nitrate, gelatin, collagen, oxidized cellulose, doxycycline, tetracycline, polidocanol, cyanoacrylate, thrombin, fibrin, chitosan, ascorbic acid, chitosan, ferric sulfate, fibrinogen, an iron oxyacid, a sodium salt of N-acyl-5-bromo(3,5-dibromo) anthranilic acid, bleomycin, clarithromycin, erythromycin, sotradecol, ankaferd, rutin, or a combination thereof.

In some embodiments, the antimicrobial agent comprises an antibiotic, antifungal, antiviral agents or combinations thereof.

In one embodiment, the antimicrobial agent comprises a metal comprising silver, copper, platinum, gold or mixtures thereof.

In another embodiment, there is a method of treating a bone defect in which the bone defect site possesses at least one cavity, the method comprising inserting an implantable matrix, the implantable matrix comprising an effective amount of at least one therapeutic agent having hemostatic and antimicrobial activity disposed in a biodegradable polymer, wherein the implantable matrix allows influx of at least progenitor, bone and/or cartilage cells therein.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the present application are approximations; the numerical values are as precise as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Additionally, unless defined otherwise or apparent from context, all technical and scientific terms used herein have the same meanings as commonly understood by one of ordinary skill in the art to which this application belongs.

Unless explicitly stated or apparent from context, the following terms or phrases have the definitions provided below:

### Definitions

It is noted that, as used in this specification and the appended claims, the singular forms “a,” “an,” and “the,” include plural referents unless expressly and unequivocally limited to one referent. Thus, for example, reference to “a matrix” includes one, two, three or more matrices.

The term “biodegradable” includes that all or parts of the matrix will degrade over time by the action of enzymes, by hydrolytic action and/or by other similar mechanisms in the human body. In various embodiments, “biodegradable” includes that a matrix (e.g., sponge, sheet, etc,) can break down or degrade within the body to non-toxic components after or while a therapeutic agent has been or is being released. By “bioerodible” it is meant that the matrix will erode or degrade over time due, at least in part, to contact with substances found in the surrounding tissue, fluids or by cellular action. By “bioabsorbable” or “bioresorbable” it is meant that the matrix will be broken down and absorbed within the human body, for example, by a cell or tissue. “Biocompatible” means that the matrix will not cause substantial tissue irritation or necrosis at the target tissue site.

The term “mammal” refers to organisms from the taxonomy class “mammalian,” including but not limited to humans, other primates such as chimpanzees, apes, orangutans and monkeys, rats, mice, cats, dogs, cows, horses, etc.

The term “resorbable” includes biologic elimination of the products of degradation by metabolism and/or excretion over time, for example, usually months.

The term “particle” refers to pieces of a substance of all shapes, sizes, thickness and configuration such as fibers, threads, narrow strips, powder, thin sheets, chips, shards, etc., that posses regular, irregular or random geometries. In some embodiments, the particles are elongated having more length than width (e.g., long and slender particles). It should be understood that some variation in dimension will occur in the production of the particles and particles demonstrating such variability in dimensions are within the scope of the present application.

The term “target tissue site” is intended to mean the location of the tissue to be treated. Typically the placement site of the matrix will be the same as the target site to provide for optimal targeted drug delivery. However, the present application also contemplates positioning the matrix at a placement site at or near the target site such that the therapeutic agent can be delivered to the surrounding vasculature, which carries the agent to the desired nearby target site. As used herein, the term “at or near” includes embodiments where the placement site and target site are within close proximity (e.g., within about 1 mm to 5 cm).

The term “autograft” as utilized herein refers to tissue that is extracted from the intended recipient of the implant.

The term “allograft” as utilized herein refers to tissue intended for implantation that is taken from a different member of the same species as the intended recipient.

The term “xenogenic” as utilized herein refers to material intended for implantation obtained from a donor source of a different species than the intended recipient. For example, when an implant is intended for use in an animal such as a horse (equine), xenogenic tissue of, e.g., bovine, porcine, caprine, etc., origin may be suitable.

The term “transgenic” as utilized herein refers to tissue intended for implantation that is obtained from an organism that has been genetically modified to contain within its genome certain genetic sequences obtained from the genome of a different species. The different species is usually the same species as the intended implant recipient but such limitation is merely included by way of example and is not intended to limit the disclosure here in anyway whatsoever.

The expressions “whole bone,” “fully demineralized bone” and “substantially fully mineralized bone” refer to bone containing its full or substantially full, original mineral content that can be used. The expression “substantially fully dernineralized bone” as utilized herein refers to bone containing less than about 8% of its original mineral context. In some embodiments, the implantable matrix comprises from about 1 to about 99% fully demineralized. or substantially fully demineralized bone. In some embodiments, the implantable matrix comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% fully demineralized or substantially demineralized bone.

The expression “demineralized bone” includes bone that has been partially, fully, segmentally or superficially (surface) demineralized. In some embodiments, the implantable matrix comprises from about 1 to about 99% demineralized bone. In some embodiments, the implantable matrix comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% demineralized bone.

In some embodiments, the implantable matrix comprises a combination of fully demineralized or substantially fully demineralized bone and dernineralized bone.

A “therapeutically effective amount” or “effective amount” is such that when administered, the therapeutic agent results in alteration of the biological activity, such as, for example, reduction of blood loss or hemorrhaging, retardation of microbial growth, promotion. of bone, cartilage and/or other tissue (e.g., vascular tissue) growth, inhibition of inflammation, reduction or alleviation of pain, improvement in the condition through inhibition of an immunologic response, etc. The dosage administered to a patient can be as single or multiple doses depending upon a variety of factors, including the therapeutic agent's (or drug's) administered pharmacokinetic properties, the route of administration, patient conditions and characteristics (sex, age, body weight, health, size, etc.), extent of symptoms, concurrent treatments, frequency of treatment and the effect desired. In some embodiments the implantable matrix is designed for sustained release. In some embodiments, the implantable matrix comprises an effective amount of a growth factor.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. In some embodiments, the matrix releases from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70% of the one or more therapeutic agent(s) within the first 12, 24, or 48 hours.

The phrases “prolonged release”, “sustained release” or “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the matrix and/or component thereof, or as the result of metabolic transformation or dissolution of the therapeutic agent(s) or conjugates of therapeutic agent(s). The release need not be linear and can be pulse type dosing.

The “matrix” of the present application is utilized as a scaffold for bone and/or cartilage repair, regeneration, and/or augmentation. Typically, the matrix provides a 3-D matrix of interconnecting pores, which acts as a pliant scaffold for cell migration. The morphology of the matrix guides cell migration and cells are able to migrate into or over the matrix, respectively. The cells then are able to proliferate and synthesize new tissue and form bone and/or cartilage. In some embodiments, the matrix is resorbable.

In some embodiments, the matrix can be shaped. The term “shaped” includes that the matrix is formed into sheets, plates, disks, cones, pins, screws, tubes, teeth, bones, portion of bone, wedges, cylinders, threaded cylinders, and the like, as well as more complex geometric configurations. In some embodiments, the matrix or a surface of the matrix comprises layers. In some embodiments, the matrix or a surface of the matrix comprises at least 1, 2, 3, 4, 5, 6, 7, 8, 9 or 10 layers. In some embodiments, the layers are composed of the same or different materials than the matrix or a surface of the matrix.

The term “aggregate” as applied to an aggregate of particles in the matrix refers to particles that adhere to each other where they clump to each other in a mass, e.g., by use of a biocompatible binder or adhesive. in some embodiments, the particles do not aggregate where they contact each other by overlapping with each other but remain separate (e.g., tether each other).

The terms “treating” and “treatment” when used in connection with a disease or condition refer to executing a protocol that may include a repair procedure (e.g., osteochondral repair procedure), administering one or more matrices to a patient (human or other mammal), in an effort to alleviate signs or symptoms of the disease or condition or immunological response, Alleviation can occur prior to signs or symptoms of the disease or condition appearing, as well as after their appearance. Thus, treating or treatment includes preventing or prevention of disease or undesirable condition. In addition, treating, treatment, preventing or prevention do not require complete alleviation of signs or symptoms, does not require a cure, and specifically includes protocols that have only a marginal effect on the patient, in some embodiments, the implantable matrix can be used to treat subchondral, osteochondral, hyaline cartilage and/or condyle defects.

The term. “subchondral” includes an area underlying joint cartilage, The term. “subchondral bone” includes a very dense, but thin layer of bone just below a zone of cartilage and above the cancellous or trabecutar bone that forms the bulk of the bone structure of the “Osteochondral” includes a combined area of cartilage and bone where a lesion or lesions can occur. “Osteochondral defect” includes a lesion, which is a composite lesion of cartilage and subchondral bone, “Hyaline cartilage” includes cartilage containing groups of isogenous chondrocytes located within lacunae cavities which are scattered throughout an extracellular collagen matrix. A “condyle” includes a rounded articular surface of the extremity of a bone.

The matrix may be osteogenic. The term “osteogenic” as used herein includes the ability of the matrix to enhance or accelerate the growth of new bone tissue by one or more mechanisms such as osteogenesis, osteoconduction and or osteoinduction. In some embodiments, the matrix is osteogenic and can be delivered to other surgical sites, particularly sites at which bone growth is desired. These include, for instance, the repair of spine (e.g., vertebrae fusion) cranial defects, iliac crest back-filling, acetabular defects, in the repair of tibial plateau, long bone defects, spinal site defects or the like. Such methods can be used to treat major or minor defects in these or other bones caused by trauma (including open and closed fractures), disease, or congenital defects, for example.

The matrix may be osteoinductive. The term “osteoinductive” as used herein includes the ability of a substance to recruit cells from the host that have the potential for forming new bone and repairing hone tissue. Most osteoinductive materials can stimulate the formation of ectopic bone in soft tissue.

The matrix may be osteoconductive. The term “osteoconductive” as utilized herein includes the ability of a non-osteoinductive substance to serve as a suitable template or substrate along which bone may grow.

The matrix may be implantable. The term “implantable” as utilized herein refers to a biocompatible device retaining potential for successful placement within a mammal. The expression “implantable device” and expressions of like import as utilized herein refers to any object implantable through surgery, injection, or other suitable means whose primary function is achieved either through its physical presence or mechanical properties.

The term “carrier” includes a diluent, adjuvant, buffer, excipient, or vehicle with which a composition can be administered. Carriers can include sterile liquids, such as, for example, water and oils, including oils of petroleum, animal, vegetable or synthetic origin, such as, for example, peanut oil, soybean oil, mineral oil, sesame oil, or the like. The growth factor may include a carrier.

The term “excipient” includes a non-therapeutic agent added to a pharmaceutical composition to provide a desired consistency or stabilizing effect. Excipients for parenteral formulations, include, for example, oils (e.g., canola, cottonseed, peanut, safflower, sesame, soybean), fatty acids and salts and esters thereof (e.g., oleic acid, stearic acid, palmitic acid), alcohols (e.g., ethanol, benzyl alcohol), polyalcohols (e.g., glycerol, propylene glycols and polyethylene glycols, e.g., PEG 3350), polysorbates (e.g., polysorbate 20, polysorbate 80), gelatin, albumin (e.g., human serum albumin), salts (e.g., sodium chloride), succinic acid and salts thereof (e.g., sodium succinate), amino acids and salts thereof (e.g., alanine, histidine, glycine, arginine, lysine), acetic acid or a salt or ester thereof (e.g., sodium acetate, ammonium acetate), citric acid and salts thereof (e.g., sodium citrate), benzoic acid and salts thereof, phosphoric acid and salts thereof (e.g., monobasic sodium phosphate, dibasic sodium phosphate), lactic acid and salts thereof, polylactic acid, glutamic acid and salts thereof (e.g., sodium glutamate), calcium and salts thereof (e.g., CaCl₂, calcium acetate), phenol, sugars (e.g., glucose, sucrose, lactose, maltose, trehalose), erythritol, arabitol, isomalt, lactitol, maltitol, mannitol, sorbitol, xylitol, nonionic surfactants (e.g., TWEEN 20, TWEEN 80), ionic surfactants (e.g., sodium dodecyl sulfate), chlorobutanol, DMSO, sodium hydroxide, glycerin, m-cresol, imidazole, protamine, zinc and salts thereof (e.g, zinc sulfate), thimerosal, methylparaben, propylparaben, carboxymethylcellulose, chlorobutanol, or heparin. The growth factor may include an excipient. In some embodiments, the pharmaceutical composition comprises a matrix, consists essentially of a matrix, or consists of a matrix. In some embodiments, the pharmaceutical composition is a bone implant. In one embodiment, the pharmaceutical composition is a conformable bone implant.

The term “lyophilized” or “freeze-dried” includes a state of a substance that has been subjected to a drying procedure such as lyophilization, where at least 50% of moisture has been removed. The matrix, antimicrobial, hemostatic agent, and/or combination thereof may be lyophilized or freeze-dried.

A “preservative” includes a bacteriostatic, bacteriocidal, fungistatic or fungicidal compound that is generally added to formulations to retard or eliminate growth of bacteria or other contaminating microorganisms in the formulations. Preservatives include, for example, benzyl alcohol, phenol, benzalkonium chloride, m-cresol, thimerosol, chlorobutanol, methylparaben, propylparaben and the like. Other examples of pharmaceutically acceptable preservatives can be found in the USP. The growth factor and/or matrix may have preservatives or be preservative free. In embodiments according to this disclosure, the preservative is not a therapeutic agent. In some embodiments, the preservative is present in an amount less than a therapeutically effective amount, however the preservative functions to preserve the implantable formulation, e.g., during storage prior to implantation.

Reference will now be made in detail to certain embodiments of the present application. While the application will be described in conjunction with the illustrated embodiments, it will be understood that they are not intended to limit the application to those embodiments. On the contrary, the application is intended to cover all alternatives, modifications, and equivalents that may be included within the application as defined by the appended claims.

Implantable matrices are provided that aid in the reduction of operative and post-operative bleeding and also kill and/or inhibit microbial growth. By using therapeutic agent(s) having hemostatic and antimicrobial activity in association with the matrix, the problems of excessive bleeding and microbial infection during and/or after surgery are addressed.

In some embodiments, the matrices provided include therapeutic agent(s) having hemostatic and antimicrobial activity that do not compromise the bioactivity of the matrix to induce or permit new tissue growth, e.g., new bone growth. In embodiments according to this disclosure, the osteogenic activity of the matrix is not reduced by the presence of the therapeutic agent(s).

In one aspect, an implantable matrix is provided that comprises at least one therapeutic agent having hemostatic and antimicrobial activity, wherein the implantable matrix is configured to be implanted into a bone defect.

In some embodiments, implantable matrices and methods are provided that retain the therapeutic agent(s) at or near the bone defect (e.g., fracture, void, etc.) to facilitate healing of the bone defect and avoid adverse local tissue reactions to the therapeutic agent(s). In some embodiments, the implantable matrices provided are osteoconductive and can be directly injected into the bone defect and allow gaps and fractures to be filled with new bridging bone faster.

In one embodiment the implantable matrices and methods allow easy delivery to the target tissue site (e.g., fracture site, synovial joint at or near the spinal column, etc.) using a flowable matrix that hardens upon contact with the target tissue. In this way, accurate and precise implantation of the matrix in minimally invasive procedure can be accomplished.

The headings below are not meant to limit the disclosure in any way; embodiments under any one heading may be used in conjunction with embodiments under any other heading.

In some embodiments, the at least one therapeutic agent comprises a hemostatic agent and an antimicrobial agent or it can be a single agent with both antimicrobial and hemostatic properties, such agents include, but are not limited to for example, silver nitrate, gelatin, collagen, oxidized cellulose, doxycycline, tetracycline, polidocanol, cyanoacrylate, thrombin, fibrin, chitosan, ascorbic acid, chitosan, ferric sulfate, fibrinogen, an iron oxyacid, a sodium salt of N-acyl-5-bromo(3,5-dibromo) anthranilic acid, bleomycin, clarithromycin, erythromycin, sotradecol, ankaferd, rutin, or a combination thereof.

### Hemostatic Agent

In some embodiments, the hemostatic agent is embedded within the matrix or the hemostatic agent may be disposed in or on one or more surfaces of the implantable matrix, or both. In some embodiments, the hemostatic agent may include more than one component. In one embodiment, the hemostatic agent can include different components that are separately combined with the matrix that together provide the matrix with hemostatic activity.

The hemostatic agent described herein provides and maintains effective hemostasis when applied to a target tissue site requiring hemostasis. Effective hemostasis, as used herein, is the ability to control and/or abate mild to moderate bleeding within an effective time, as recognized by those skilled in the art of hemostasis. Further indications of effective hemostasis may be provided by governmental regulatory standards and the like. Hemostatic agents, for purposes of this application, include agents that have a hemostatic effect, more preferably, slow, impede and eventually stop bleeding at the site of the injury or surgery. One method for producing a hemostatic effect at the site of an injury is to introduce one or more hemostatic agents (e.g., thrombin, fibrinogen, silver nitrate, etc.) to produce the desired hemostatic effect.

For example, when the hemostatic agent comprises thrombin and/or fibrinogen in the matrix, it maybe animal derived, human, or may be recombinant. The thrombin activity may be in the range of about 20 to 500 IU/cm², about 20 to 200 IU/cm², or about 50 to 200 IU/cm². The fibrinogen activity of the matrix may be in the range of about 2 to 15 mg/cm², about 3 to 12 mg/cm², or about 5 to 10 mg/cm².

The hemostatic agent can be chitosan lactate, chitosan salicylate, chitosan pyrrolidone carboxylate, chitosan itaconate, chitosan niacinate, chitosan formate, chitosan acetate, chitosan gallate, chitosan glutamater, chitosan maleate, chitosan aspartate, chitosan glycolate, quaternary amine substituted chitosan or salts thereof. The chitosan can be in the form of granules or particles having a density between about 0.25 to 0.60 g/cm³ and a diameter of about 0.5 mm to about 0.9 mm, These particles can be layered or disposed uniformly throughout the matrix.

The hemostatic agent can be an alginate, such as for example, sodium alginate, potassium alginate, magnesium alginate, calcium alginate, or aluminum alginate.

In some embodiments, the hemostatic agent can be hydroxyethyl cellulose, hydroxypropyl cellulose, methyl cellulose, hydroxypropyl methyl cellulose, or hydroethyl methyl cellulose.

In some embodiments, the antimicrobial and/or hemostatic agent can be in nanoparticle form and disposed in or on the matrix homogenously throughout the matrix, layered on or in the matrix, or disposed on layers at the surface of the matrix. As used herein, the terms “nanoparticle” and “nanoscale particles” are used interchangeably and refer to a nanoscale particle with a size that is measured in nanometers, for example, a nanoscopic particle that has at least one dimension of from about 1000, 950, 900, 850, 800, 750, 700, 650, 600, 550, 500, 450, 400, 350, 300, 250, 200, 150, 100, 50, 25, 10, to about 5 nm. Examples of nanoparticles include nanobeads, nanofibers, nanohorns, nano-onions, nanorods, and nanoropes.

In some embodiments, the antimicrobial and/or hemostatic agent can be in microparticle form and disposed in or on the matrix homogenously throughout the matrix, layered on or in the matrix, or disposed on layers at the surface of the matrix. As used herein, the term “microparticle” and “microscale particles” are used interchangeably and refers to a microscale particle with a size that is measured in micrometers, for example, a microscale particle that has at least one dimension of from about 1.0, 1.5, 2.0, 2.5, 3.0, 3.5, 4.0, 4.5, 5.0, 5.5, 6.0, 6.5, 7.0, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0 20.5, 21.0, 21.5, 22.0, 22.5, 23.0, 23.5, 24.0, 24.5, 25.0, 25.5, 26.0, 26.5, 27.0, 27.5, 28,0, 28,5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0 45.5, 46.0, 46.5, 47.0, 47.5, 48.0, 48.5, 49.0, 49.5 to about micrometers.

In some embodiments, the antimicrobial and/or hemostatic agent comprises silver. For example, silver nitrate, silver chloride, silver dioxide, silver sulfate, silver calcium phosphate, silver carboxymethylcellulose, silver sulfadiazine, silver zirconium phosphate, silver glass, silver zeolite complex, nano silver or a combination thereof.

In some embodiments, the antimicrobial and/or hemostatic agent comprises from about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 071, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36,5, 37,0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, to about 50.0% by w/w, w/v, or v/v of the total volume or weight of the matrix.

In some embodiments, the hemostatic agent includes a biocompatible material for promoting blood clotting. The clot producing material, according to certain embodiments, may include polyethylene glycol (PEG), aluminum, hydroxyapatite, which may be unsintered, absorbents, absorbent DBM that has been treated to alter the surface tension of surrounding liquids to provide for rapid water uptake into the bone, a hydroscopic agent, a surface tension reducing material, and/or a substance capable of inducing protein precipitation, such as those materials capable of removing the water of solvation from protein. In another embodiment, the biocompatible material for blood clotting can include bone, biocompatible polymers, or combinations thereof that are configured as wicking materials such as capillary tubes, small fibers, or U-shaped materials that allow blood to clot upon wicking.

In another embodiment, the hemostatic agent may include a sealant. In some embodiments, the sealant may take the form of a waxy, sticky substance, including lipids, PEG, lecithin, saccharides such as polysaccharides, fatty acids, including high molecular weight filly acids, other suitable sealants, or combinations of these. Glycerol may be added to the waxy material in some embodiments.

In one embodiment, the hemostatic agent comprises substantially water-free demineralized bone and lecithin. In some embodiments, the bone may be in a concentration high enough to establish substantial contiguity of the bone, which may be done using bone fibers or bone particles. In some embodiments, the hemostatic agent comprises from about 1 to about 99% water-free demineralized bone. In some embodiments, the hemostatic agent comprises from about 1 to about 75%, from about 1 to about 50%, from about 1 to about 25%, from about 1 to about 10%, from about 10 to about 30%, from about 20 to about 50%, from about 30 to about 70%, from about 15 to about 75%, from about 30 to about 50%, from about 60 to about 80% water-free demineralized bone. In some embodiments, the hemostatic agent may also include a surface tension reduction material. For example, PEG may be used, for example, to provide for increased uptake of water into the bone.

In embodiments utilizing bone materials, any suitable type of bone materials can be used, including substantially fully demineralized bone, partially demineralized bone, surface demineralized bone, or nondemineralized bone, or mineralized bone.

In some embodiments, the surface tension reduction material may include glycerol, non-crystalline starch, amphipathic zwitterions, a polyalcohol, and/or aluminum sulfate, other suitable materials, or combinations of these. In some embodiments, ethanol may be used.

In some embodiments, a protein precipitating agent may be added to the hemostatic agent including ammonium sulfate, PEG, a hydrogel, unsintered hydroxyapatite, calcium phosphate, other suitable agents, or combinations of these, Other embodiments may include as a clot producing material that absorbs water from blood, leading to clot formation. In another embodiment, the biocompatible material for promoting blood clotting includes demineralized bone matrix and a hydrostatic agent, in which case the biocompatible material may take the form of a sheet, a powder, a matrix, a paste, a wax, a gel, or other suitable form. A hydrostatic agent used in accordance with particular embodiments may include the use of waxes, solid fatty acids or derivatives, non-crystalline starches, PEG, or combinations thereof.

In some embodiments, the hemostatic agent is a biocompatible material fur promoting blood clotting that includes demineralized bone matrix, a protein precipitating agent, and a material that promotes water uptake by the demineralized bone. Because PEG affects both protein precipitation and promotes water uptake by DBM, according to certain embodiments, PEG may be used as either a protein precipitating agent or as a material that promotes water uptake by DBM.

In some embodiments, the biocompatible material for promoting blood clotting can be prepared by mixing lyophilized demineralized bone matrix and PEG. The demineralized bone matrix and PEG may be in a ratio of about 1:9, about 3:2, a ratio in between, or any other suitable ratio. In some embodiments, the demineralized bone matrix and the PEG may be in a ratio of about 1:1, 1:2, 1:3, 1:4, 1:5, 1.:6, 1:7, 1:8, 1:9, 9:1, 9:2, 9:3, 9:4, 9:5, 9:6, 9:7, 9:8 or 9:9. The mix may further include about four parts water to a mixture of about three parts dernineralized bone matrix to about two parts PEG. According to certain embodiments, the PEG may be melted in order to facilitate blending with the DBM. The mixture may be lyophilized and/or the demineralized bone matrix may be lyophilized, according to some embodiments.

In some embodiments, the biocompatible material for promoting blood clotting is prepared by mixing demineralized bone matrix and aluminum sulfate, freezing the mixture, and lyophilizing the mixture. In another embodiment, the biocompatible material for promoting blood clotting is prepared by mixing demineralized bone matrix and lecithin. The mixture may be heated and/or smoothed. Furthermore, the demineralized bone matrix may be smoothed. In other certain embodiments, the mixture may further include a carrier and a preservative.

In some embodiments, the hemostatic agent can include, but is not limited to, prothrombin, thrombin, fibrin, fibronectin, Factor X/Xa, Factor VII/VIIa, Factor IX/IXa, Factor XI/XIa, Factor XII/XIIa, factor XIII, factor VIII, vitronectin, tissue factor, proteolytic enzyme obtainable from snake venom such as batroxobin, von Willebrand Factor, plasminogen activator inhibitor, platelet activating agents, synthetic peptides having hemostatic activity, collagen particles, derivatives of the above or any combination thereof. These hemostatic agents can enhance clotting. In some embodiments, the hemostatic agent comprises gelatins, collagens, oxidized celluloses, thrombin and fibrin sealants, chitosan, synthetic glues, glutaraldehyde-based glues, or a combination thereof. In some embodiments, the hemostatic agent includes collagen particles. In embodiments, the collagen particles have a mean diameter within the range of from about 5 microns to about 1000 microns, or from about 50 microns to about 500 microns. In some embodiments, the collagen particles have a mean diameter of about 100 microns to about 200 microns.

Collagen particles can be obtained from various collagen sources including human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited collagen types. In some embodiments, the collagen comprises hetero- or homo-trimers of human collagen type I, human collagen type II, human collagen type III, or combinations thereof.

In some embodiments, the hemostatic agent is present in a therapeutically effective amount. In one embodiment, the hemostatic agent is present in an amount up to 10 wt %, based on the total weight of the matrix. In another embodiment, the hemostatic agent is present in an amount from about 0.1 to about 10 wt %, based on the weight of the matrix. In some embodiments, the amount of hemostatic agent is not greater than 9, or 8, or 7, or 6, or 5 wt %, based on the weight of the matrix. In some embodiments, the hemostatic agent is present in an amount of at least 0.2, or 0.3, or 0.4, or 0.5 wt %, based on the weight of the matrix. In one embodiment, the hemostatic agent is present from about 0.1 to about 5 wt %, based on the weight of the matrix. In another embodiment, the hemostatic agent is present from about 1 to about 5 wt %, based on the weight of the matrix.

In some embodiments, the hemostatic agent comprises gelatins, collagens, oxidized celluloses, thrombin and fibrin sealants, chitosan, synthetic glues, glutaraldehyde-based glues, derivatives thereof or a combination thereof.

### Antimicrobial Agent

In some embodiments, the antimicrobial agent is embedded within the matrix or the antimicrobial agent may be disposed in or on one or more surfaces of the implantable matrix, or both. In some embodiments, the antimicrobial agent may include more than one component. In one embodiment, the antimicrobial agent can include different components that are separately combined with the matrix that together provide the matrix with antimicrobial activity.

In some embodiments, the antimicrobial agent is selected from the group consisting of an antibiotic agent, antifungal agent, antiviral agent or combinations thereof.

In the present application, the term “antimicrobial activity” includes activities for suppressing proliferation of a microorganism, eliminating microorganisms, reducing the number thereof or decolonize, or killing the microorganisms in or at the matrix and/or target tissue site. Microorganisms include viruses, bacteria, fungi, spores, yeast or the like. Examples of microorganisms include gram-negative bacteria such as Escherichia, Salmonella, Listeria, Cronobacter, Klebsiella, Proteus mirabilis, Pseudomonas bacteria , gram-positive bacteria such as Staphylococcus epidermidis, Staphylococcus aureus, methicillin-resistant Staphylococcus epidermidis, methicillin-resistant Staphylococcus aureus, Streptococcus pyogenes, fungi such as Candida albican, or a combination thereof.

An effective amount of antimicrobial in the matrix is that amount for suppressing proliferation of a microorganism (bacteriostatic), eliminating microorganisms, reducing the number thereof or decolonizing, or killing the microorganisms (bactericidal) in or at the matrix and/or target tissue site. The effective amount can, in some embodiments, be measured in vitro by measuring the microorganism's MIC (minimum inhibitory concentration) when challenged with the antimicrobial. The MIC will vary depending on the organism being challenged with the antimicrobial. In some embodiments, the MIC can be from about 0.062, 0.03, 0.016, 0.008, 0.004, 0.002, 0.001, to about 0.0005 in a broth dilution with the antimicrobial.

In another general aspect, there is provided a matrix having an antimicrobial agent in layer upon layer stacked on the surface of the matrix.

In some embodiments, the antimicrobial and/or hemostatic agent can be in nanoparticle form and disposed in or on the matrix homogenously throughout the matrix, layered on or in the matrix, or disposed on layers at the surface of the matrix. As used herein, the terms “nanoparticle” and “nanoscale particles” are used interchangeably and refer to a nanoscale particle with a size that is measured in nanometers, for example, a nanoscopic particle that has at least one dimension of from about 1000, 950, 900, 850, 800, 750, 700, 650, 600, 550, 500, 450, 400, 350, 300, 250, 200, 150, 100, 50, 25, 10, to about 5 nm, Examples of nanoparticles include nanobeads, nanofibers, nanohorns, nano-onions, nanorods, or nanoropes.

In some embodiments, the antimicrobial and/or hemostatic agent can be in microparticle form and disposed in or on the matrix homogenously throughout the matrix, layered on or in the matrix, or disposed on layers at the surface of the matrix. As used herein, the term “microparticle” and “microscale particles” are used interchangeably and refers to a microscale particle with a size that is measured in micrometers, fir example, a microscale particle that has at least one dimension of from about 1.0, 1.5, 2.0, 2.5, 3.0, 3.5, 4.0, 4.5, 5.0, 5.5, 6.0, 6.5, 7.0, 7.5 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27,0, 27.5, 28.0, 28.5, 29.0, 29.5, 30.0, 30.5, 31.0, 31.5, 32.0, 32,5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5 to about 50 micrometers.

In some embodiments, the antimicrobial and/or hemostatic agent comprises silver. For example, silver nitrate, silver chloride, silver dioxide, silver sulfate, silver calcium phosphate, silver carboxymethylcellulose silver sulfadiazine, silver zirconium phosphate, silver glass, silver zeolite complex, nano silver or a combination thereof.

In some embodiments, the antimicrobial and/or hemostatic agent comprises from about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 071, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5 19.0, 19.5 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44,5, 45.0, 45.5, 46.0, 46.5, 47.0, 47.5, 48.0, 48.5, 49.0, 49.5, to about 50.0% by w/w, w/v, or v/v of the total volume or weight of the matrix.

In some embodiments, the antimicrobial agent can include by way of example and not limitation, antiseptic agents, antibacterial agents; quinolones and in particular fluoroquinolones (e.g., norfloxacin, ciprofloxacin, lomefloxacin, ofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), polymyxins, tetracyclines (minocycline, doxycycline, tetracycline, etc.), or combinations thereof.

In embodiments, the antimicrobial agent can include one or more of triclosan, also known as 2,4,4′-trichloro-2′-hydroxydiphenyl ether, chlorhexidine and its salts, including chlorhexidine acetate, chiorhexidine &collate, chlorhexidine hydrochloride, and chiorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof.

Examples of antimicrobial agents include, by way of illustration and not limitation, acedapsone; acetosulfone sodium; alamecin; alexidine; amdinocillin; amdinociilin pivoxil; amicycline; amifloxacin; amifloxacin mesylate; amikacin; amikacin sulfate; aminosalicylic acid; aminosalicylate sodium; amoxicillin; amphomycin; ampicillin sodium; apalcillin sodium; apramycin; aspartocin; astromicin sulfate; avilamycin; avoparcin; azithromycin; aziocillin; aziocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole palate; cefamandole sodium; cefaparole; cefatrizine; cefazaflur sodium; cefazolin; cefazolin sodium; cetbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; cournermycin sodium; cyclacillin; cycloserine; dalfopristin; dapsone; daptomycin; demeclocycline; demeclocycline hydrochloride; demecycline; denofungin; diaveridine; dicloxacillin sodium; dihydrostreptomycin sulfate; dipyrithione; dirithrotnycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; triethenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natamycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

Antiviral agents can include, but are not limited to, vidarabine, acyclovir, famciclovir, valacyclovir, gancyclovir, valganciclovir, nucleoside-analog reverse transcriptase inhibitors (such as AZT (zidovudine), ddI (didanosine), ddC (zalcitabine), d4T (stavudine), and 3TC (lamivudine)), nevirapine, delavirdine, protease inhibitors (such as saquinavir, ritonavir, indinavir, and nelfinavir), ribavirin, amantadine, rimantadine, neuraminidase inhibitors (such as zanamivir and oseitamivir), pleconaril, cidofbvir, foscarnet, and/or interferons.

In some embodiments, the antimicrobial agent can comprises a metal such as for example silver, such as for example, silver ions, metallic silver, silver salt, copper, platinum, gold or mixtures thereof. The metal may be present in about 1%, 2%, 3%, 4%, 5% by weight based on the total weight of the matrix. In some embodiments, the silver can be in combination with chitosan or the chitosan can be alone in the composition to provide antimicrobial activity. For example, freeze-dried chitosan acetate incorporating silver nanoparticles can provide antimicrobial properties to the matrix.

In one embodiment, the antimicrobial agent comprises a metal comprising silver, copper, platinum, gold, a salt thereof, or mixtures thereof. In one embodiment, the metal can be combined with a carrier or support, e.g., a solid zeolite support.

In some embodiments, the antimicrobial agent is present in a therapeutically effective amount. In one embodiment, the antimicrobial agent is present in an amount up to 5 wt %, based on the total weight of the matrix. In one embodiment, the antimicrobial agent is present in an amount up to 1 wt %, based on the total weight of the matrix. In another embodiment, the antimicrobial agent is present in an amount from about 0.01 to about 1 wt %, based on the weight of the matrix, in some embodiments, the amount of antimicrobial agent is not greater than 0.9, or 0.8, or 0.7, or 0.6, or 0.5 wt %, based on the weight of the matrix. In some embodiments, the antimicrobial agent is present in an amount of at least 0.02, or 0.03, or 0.0.4, or 0.05 wt %, based on the weight of the matrix. in one embodiment, the antimicrobial agent is present from about 0.01 to about 0.5 wt %, based on the weight of the matrix. In another embodiment, the antimicrobial agent is present from about 0.1 to about 0.5 wt %, based on the weight of the matrix.

### Multifunctional Therapeutic Agent

In some embodiments, the at least one therapeutic agent comprises a multifunctional therapeutic agent having both hemostatic and antimicrobial activity.

In some embodiments, the multifunctional therapeutic agent is embedded within the matrix or the multifunctional therapeutic agent may be disposed in or on one or more surfaces of the implantable matrix, or both.

In embodiments, the multifunctional therapeutic agent may be derived from or contain one or more of the hemostatic or antimicrobial agents discussed above, provided that the multifunctional therapeutic agent has both hemostatic and antimicrobial activity. In embodiments, the hemostatic or antimicrobial agents, as described above, may be modified from their typical form as an agent having a primary or sole function as either a hemostatic agent or an antimicrobial agent to provide a multifunctional therapeutic agent having both hemostatic and antimicrobial activity. In some embodiments, the modification to the hemostatic or antimicrobial agent(s) can be the amount, e.g., concentration, or physical form, e.g., particle size, chemical structure, surface characteristics, or a combined form with other materials, such as a carrier or support material, that provides a multifunctional therapeutic agent having both hemostatic and antimicrobial activity. In embodiments, the modification to the hemostatic or antimicrobial agent(s) results in a multifunctional therapeutic agent having both hemostatic and antimicrobial activity that differs from the activity of a simple combination of similar amounts of individual hemostatic and/or antimicrobial agents.

In embodiments, the multifunctional therapeutic agent is present in a therapeutically effective amount that provides both hemostatic and antimicrobial activity. In one embodiment, the multifunctional therapeutic agent is present in an amount up to 25 wt %, based on the total weight of the matrix. in another embodiment, the multifunctional therapeutic agent is present in an amount from about 1 to about 25 wt %, based on the weight of the matrix. In some embodiments, the amount of multifunctional therapeutic agent is not greater than 2.4, or 23, or 22, or 21, or 20 wt %, based on the weight of the matrix. In some embodiments, the multifunctional therapeutic agent is present in an amount of at least 2, or 3, or 4, or 5 wt %, based on the weight of the matrix. In one embodiment, the multifunctional therapeutic agent is present from about 5 to about 20 wt %, based on the weight of the matrix. In another embodiment, the antimicrobial agent is present from about 10 to about 20 wt %, based on the weight of the matrix. In some embodiments, the multifunctional therapeutic agent is present from about 5 to about 15 wt %, based on the weight of the matrix.

In one embodiment, the multifunctional therapeutic agent is silver nitrate particles having an average particle size greater than 1 micron. In some embodiments, the silver nitrate particles have an average particle size greater than 2, or 3, or 4, or 5 microns. In one embodiment, the silver nitrate particles have an average particle size greater than 10 microns.

In one embodiment, the multifunctional therapeutic agent is a chitosan based material. In one embodiment, the multifunctional therapeutic agent is a chitosan niacinamide ascorbate salt.

In another aspect, a conformable bone implant is provided having hemostatic and antimicrobial properties. in some embodiments, the conformable bone implant comprises an implantable matrix comprising at least one therapeutic agent having hemostatic and antimicrobial activity.

In one embodiment, the conformable bone implant comprises a multifunctional therapeutic agent having both hemostatic and antimicrobial activity.

In yet another aspect, a method of treating a bone defect in which the bone defect site possesses at least one cavity is provided. In some embodiments, the method comprises inserting an implantable matrix into the defect, the matrix comprising at least one therapeutic agent having hemostatic and antimicrobial activity, wherein the matrix allows influx of at least progenitor, bone and/or cartilage cells therein.

In some embodiments, an implantable matrix is provided configured to fit at or near a target tissue site, the matrix comprising a biodegradable polymer. In one embodiment, the matrix also comprises a plurality of particles embedded within the polymer. The particles can be entangled with each other and/or embedded in the polymer uniformly or randomly. In some embodiments, the matrix allows influx of at least progenitor, bone and/or cartilage cells therein.

In some embodiments, the matrix may comprise a growth factor. In some embodiments, the growth factor (e.g., rhBMP-2) will be more evenly distributed throughout the interior of the matrix and facilitate more uniform bone growth throughout the whole matrix. In some embodiments, the growth factor (e.g., rhBMP-2) is temporarily retained within the matrix so as to limit new bone formation to within the matrix.

### Matrix

The matrix provides a tissue scaffold for the cells to guide the process of tissue formation in vivo in three dimensions. The morphology of the matrix guides cell migration and cells are able to migrate into or over the matrix. The cells then are able to proliferate and synthesize new tissue and form bone and/or cartilage. In some embodiments, one or more tissue matrices are stacked on one another. In some embodiments, 1, 2, 3, 4, 5, 6, 7, 8, 9, 10, 11 or 12 matrices are stacked on one another.

The matrix is porous and configured to allow influx of at least bone and/or cartilage cells therein. In some embodiments, the matrix is also configured to release the therapeutic agent(s) and other bioactive agents, e.g., a growth factor. By porous it is meant that the matrix has a plurality of pores or interstitial spaces, for example a matrix having nano, micro, meso and macro porosities. In embodiments, the pores of the matrix are a size large enough to allow influx of blood, other bodily fluid, and progenitor and/or bone and/or cartilage cells into the interior to guide the process of tissue formation in vivo in three dimensions.

In some embodiments, the matrix comprises a plurality of pores. In some embodiments, at least 10% of the pores are between about 250 micrometers and about 500 micrometers at their widest points. In some embodiments, at least 20% of the pores are between about 250 micrometers and about 500 micrometers at their widest points. In some embodiments, at least 30% of the pores are between about 250 micrometers and about 500 micrometers at their widest points. In some embodiments, at least 50% of the pores are between about 250 micrometers and about 500 micrometers at their widest points, In some embodiments, at least 90% of the pores are between about 250 micrometers and about 500 micrometers at their widest points. In some embodiments, at least 95% of the pores are between about 250 micrometers and about 500 micrometers at their widest points. In some embodiments, 100% of the pores are between about 250 micrometers and about 500 micrometers at their widest points.

In some embodiments, the matrix has a porosity of at least about 30%, at least about 50%, at least about 60%, at least about 70%, at least about 90% or at least about 95%, or at least about 99%. The pores support ingrowth of cells, formation or remodeling of bone, cartilage and/or vascular tissue.

In some embodiments, the matrix is solid and has a modulus of elasticity in the range of about 1×10² to about 6×10⁵ dynes/cm², or 2×10⁴ to about 5×10⁵ dynes/cm², or 5×10⁴ dynes/cm² to about 5×10⁵ dynes/cm².

In some embodiments, the matrix may comprise a polymer having a molecular weight, as shown by the inherent viscosity, from about 0.10 dL/g to about 1.2 dig or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.55 to about 0.6 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 L/g, about 1.1 to about 1.3 dL/g, about 1.2 to about 1.4 dL/g, about 1.3 to about 1.5 dL/g, about 1.4 to about 1.6 dL/g, about 1.5 to about 1.7 L/g, about 1.6 to about 1.8 L/g, about 1.7 to about 1.9 dL/g, or about 1.8 to about 2.1 dL/g.

In various embodiments, the matrix can be designed to cause an initial burst dose of therapeutic agent (e.g., antimicrobial and/or hemostatic agent or a single agent with both properties) within the first twenty-four to forty-eight hours after implantation. “Initial burst” or “burst effect” “burst release” or “bolus dose” refers to the release of therapeutic agent from the matrix during the first twenty-four hours to forty-eight hours after the matrix comes in contact with an aqueous fluid (e.g., interstitial fluid, synovial fluid, cerebral spinal fluid, etc.). The “burst effect” is believed to be due to the increased release of therapeutic agent from the matrix. In some embodiments, the matrix has one or more burst release surfaces that releases about 10%, 15%, 20%, 25%, 30%, 35%, 45%, to about 50% of the drug over 24 or 48 hours.

In some embodiments, the therapeutically effective dosage amount (e.g., antimicrobial, hemostatic agent, or a single agent with both properties) and the release rate profile are sufficient to release the agent from the matrix for a period of at least 14 days, for example, 14-90 days, 14-30 days, 14-60 days, 21-90 days, 21-180 days; 14-210 days, or 14 days to 6 months or 1 year or longer.

In some embodiments, the matrix does not contain any growth factor. In some embodiments, the matrix does contain one or more growth factors.

In some embodiments, the matrix has a porous interior, which can hold the growth factor and because the interior is porous, the growth factor can be evenly distributed throughout the matrix when growth factor is injected into the matrix.

In some embodiments, growth factor will be held within the interior of the matrix and released into the environment surrounding the matrix (e.g., bone defect, osteochondral defect, etc.) as the matrix degrades over time.

In some embodiments, the matrix comprises biodegradable polymeric and/or non-polymeric material. For example, the matrix may comprises one or more poly (alpha-hydroxy acids), poly (lactide-co-glycolide) (PLGA.), polyiactide (PLA), poly(L-lactide), polyglycolide (PG), polyethylene glycol (PEG) conjugates of poiy (alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheiyl acetate, d-alpha tocopheryl succinate, D,L-lactide, or L-lactide-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, POE, SAM (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteratnides, polyetheretherketone, polymethylmethacrylate, silicone, hyaluronic acid, chitosan, or combinations thereof, and any random or (multi-)block copolymers such as bipolymer, terpolymer, quaterpolymer, etc., that can be polymerized from the monomers related to any of the previously-listed homo- and copolymers. It will be well understood that these an other implantable materials, or combinations thereof, may be used in certain embodiments.

In some embodiments, the matrix (e.g., exterior and/or interior) comprises collagen. Exemplary collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited. collagen types. In some embodiments, the collagen comprises hetero- or homo-trimers of human collagen type I, human collagen type IL human collagen type III, or combinations thereof.

In some embodiments, the matrix comprises collagen-containing biomaterials from the implant market which, when placed in a bone defect, provide scaffolding around which the patient's new bone and/or cartilage will grow, gradually replacing the carrier matrix as the target site heals. Examples of suitable carrier matrices may include, but are not limited to, the MasterGraft® Matrix produced by Medtronic Sofamor Danek, Inc., Memphis, Tenn.; MasterGrath® Putty produced by Medtronic Sofamor Danek, Inc., Memphis, Tenn.; Absorbable Collagen Sponge (“ACS”) produced by Integra LifeSciences Corporation, Plainsboro, N.J.; bovine skin collagen fibers coated with hydroxyapatite, e.g. Healos®. marketed by Johnson & Johnson, USA; collagen sponges, e.g. Hemostagene® marketed by Coletica S A, France, or e.g. Helisat® marketed by Integra Life Sciences Inc., USA; and Collagraft® Bone Graft Matrix produced by Zimmer Holdings, Inc., Warsaw, Ind.

Compression resistance is needed for many tissue engineering applications such as tibial plateau fractures, acetabular defects, long bone comminuted fractures, oral maxillofacial defects, spinal fusions, and cartilage subchondral defects. Compression resistant matrices will help facilitate adequate volumes of newly formed bone.

In some embodiments, the matrix is compression resistant where the matrix resists reduction in size or an increase in density when a force is applied as compared to matrices without the elongated particles disposed in it. In various embodiments, the matrix resists compression by at 10%, 15%, 20%, 25%, 30%, 35%, 40%, 45%, 50%, 55%, 60%, 65%, 70%, 75%, 80%, 85%, 90%, 95%, or more in one or all directions when a force is applied to the matrix.

In certain aspects, an implantable matrix is provided, wherein the matrix is of such a nature that it is conformable or can be readily disrupted and broken down into a conformable substance, such as a putty or paste, upon wetting with a biocompatible liquid.

In some embodiments, an implantable matrix (or medical article) is provided that is conformable or has a body that will exhibit a disruptable character such that it can be broken down by physical manipulation (e.g., manual crushing and kneading) when combined with a liquid and become conformable. Upon such maniplation, the formed material will exhibit a more conformable character than the original body, such as that provided by a putty, paste or more flowable form, depending for instance upon the amount of liquid combined with a given amount of body material solids. In certain embodiments, the conformable material e.g., formed upon wetting and disruption of the body, will be a putty exhibiting a combination of advantageous properties including malleability, cohesiveness, and shape retention.

In this regard, as used herein the term “malleable” means that the material is capable of being permanently converted from a first shape to a second shape by the application of pressure. The term “cohesive” as used herein means that the putty tends to remain a singular, connected mass upon stretching, including the exhibition of the ability to elongate substantially without breaking upon stretching. In the contest of putties containing insoluble collagen fibers or another natural or synthetic fibrous material, upon stretching, the putties can exhibit elongation, during which the existence of substantial levels of intermeshed fibers clinging to one another becomes apparent. As used herein, the term “shape-retaining” means that a putty material is highly viscous and unless acted upon with pressure tends to remain in the shape in which it is placed. On the other hand, embodiments that are based on thinner paste form materials flow more readily than putties, and thus tend to deform substantially under the force of gravity (e.g. pool or puddle) upon application to a surface. Further, in some embodiments, the matrix or implantable article can be in the form of low-viscosity, highly flowable (e.g. injectable) materials.

In some embodiments, fibrous materials, including fibrous protein materials, can be used in the implantable matrix. These include, as examples, fibers comprising collagen, elastin, fibronectin, laminin, or other similar structural, fiber-forming proteins. Insoluble, fibrous demineralized bone matrix (DBM) materials can also be used, alone or in combination with other fibrous materials disclosed herein.

In certain embodiments, the implantable matrix will be in the form of a gel upon combination with a biocompatible liquid, e.g. after being solubilized by the biocompatible liquid. Suitable gel-forming agents for these purposes include, for instance; plant extracts such as agar, ispaghula, psyllium, cydonia or ceratonia; vegetable oils such as hydrogenated castor oil; gums such as guar gum, acacia gum, ghatti gum, karaya gum, tragacanth gum or xanthan gum; synthetic and natural polysaccharides such as alkylcelluloses, hydroxyalkylcelluloses, cellulose ethers, cellulose esters, nitrocelluloses, dextrin, agar, carrageenan, pectin, furcellaran or starch or starch derivatives such as sodium starch glycolate; polysaccharides such as agar and carrageenan; polypeptides such as zein, gelatin, soluble collagen and polygeline; or mixtures of two or more of any of these or other suitable gel-forming agents. Further, in some embodiments, a gel-forming agent that is soluble in the biocompatible wetting liquid will be used in combination with another implantable matrix that is insoluble in the biocompatible liquid, e.g. an insoluble fibrous material as discussed above, to ultimately form a paste, putty or more flowable wetted implant material comprising insoluble fibers suspended or mixed with a gel substance.

In some embodiments, the gel is a hardening gel, where after the gel is applied to the target site, it hardens and allows it to conform to irregularities, crevices, cracks, and/or voids in the tissue site. For example, in some embodiments, the gel may be used to fill one or more voids in an osteolytic lesion.

In some embodiments, the gel is flowable and can ve injected, sprayed, instilled, and/or dispensed to,, on or in the target tissue site. In some embodiments, the gel has a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In embodiments, a gel is provided that hardens or stiffens after delivery. Typically, hardening gel formulations may have a pre-dosed modulus of elasticity in the range of about 1×10⁴ to about 3×10⁵ dynes/cm², or 2×10⁴ to about 2×10⁵ dynes/cm², or 5×10⁴ to about 1×10⁵ dynes/cm². The post-dosed hardening gels (after delivery) may have a rubbery consistency and have a modulus of elasticity in the range of about 1×10⁴ to about 2×10⁶ dynes/cm2, or 1×10⁵ to about 7×10⁵ dynes/cm², or 2×10⁵ to about 5×10⁵ dynes/cm².

In some embodiments, the gel comprises a viscosity enhancing agent. In some embodiments, the viscosity enhancing agent comprises mannitol, trehalose, hydroxypropyl cellulose, hydroxypropyl methylcellulose, hydroxyethyl methylcellulose, carboxymethylcellulose and salts thereof, Carbopol, poly-(hydroxyethyl-methacrylate), poly-(methoxyethylmethacrylate), poly(methoxyethoxyethylmethacrylate), polymethyl-methacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In some forms, the putty or other wetted, conformable implant material contains both insoluble collagen fibers and soluble collagen. The soluble collagen and insoluble collagen fibers can first be prepared separately, and then combined. Both the soluble collagen and the insoluble collagen fibers can be derived from bovine hides, but can also be prepared from other collagen sources (e.g. bovine tendon porcine tisues recombinant DNA techniques, fermentation etc.). Suitable collagen materials can be prepared using these or other techniques known in the literature or can be obtained from commercial sources, including for example from Kensey Nash. Corporation (Exton, Pa.) which manufactures soluble collagen known as Semed S, fibrous collagen known as Semed F, and a composite collagen known as P1076. Naturally-derived human collagen or recombinant human collagen can also be used in certain embodiments.

In some embodiments, the matrix comprises collagen-containing biomaterials from the implant market which, when placed in a bone defect provide scaffolding around which the patient's new bone and/or cartilage will grow, gradually replacing the carrier matrix as the target site heals. Examples of suitable carrier matrices may include, but are not limited to, the MasterGraft® Matrix produced by Medtronic Sofamor Danek, Inc., Memphis, Ten.; MasterGraft® Putty produced by Medtronic Sofamor Danek, Inc., Memphis, Tenn.; Absorbable Collagen Sponge (“ACS”) produced by Integra LifeSciences Corporation, Plainsboro, N.J.; bovine skin collagen fibers coated with hydroxyapatite, e.g. Healos®, marketed by Johnson & Johnson USA; collagen sponges e.g. Hemostagene® marketed by Coletica S A, France, or e.g. Helisat® marketed by Integra Life Sciences Inc. USA., Collagraft® Bone Graft Matrix produced by Zimmer Holdings, Inc., Warsaw, Ind. Osteofil® (Medtronic Sofamor Danek, Inc., Memphis, Tenn.), Allomatrix® (Wright), Grafton® (Osteotech), DBX® (MTF/Synthes), Bioset® (Regeneration Technologies), matrices consisting of mineral phases such as Vitoss® (Orthivista), Osteoset® (Wright) or mixed matrices such as CopiOs® (Zimmer), or Sunnmax Collagen Bone Graft Matrix (Sunmax). This matrix comprises the antimicrobial agent, the hemostatic agent or the combination thereof or an agent with both hemostatic and antimicrobial properties.

In one embodiment, the matrix can be packaged as a product including a container body holding an unhydrated matrix to be hydrated, and a renlovable seal operable to prevent passage of moisture into contact with the matrix. Exemplary materials to be hydrated include MasterGraft® Matrix and a MasterGraft® Putty. Exemplary hydrating fluids include blood, bone marrow, saline, water, or other fluid. The hydrating fluid may contain the therapeutic agent(s) and be used to soak the agent(s) in the matrix. For example, the therapeutic agent(s) can be applied to MasterGraft® Matrix or MasterGraft® Putty, which comprises type I bovine collagen and a calcium phosphate mineral phase composed of 15% hydroxyapatite and 85% beta-tricalcium phosphate. The matrix can be hydrated just prior to use so that, in some embodiments, it becomes a flowable material. Such a material can be injected through a cannula or other conduit into an in vivo location.

In some embodiments, the present application includes an implantable osteoconductive matrix that is in the form of a medical putty, and includes methods and materials that are useful for preparing such an osteoconductive medical putty. In some embodiments, the medical putties possess a combination of properties including a mineral content, malleability, cohesiveness, and shape retention. For example, when the matrix is implanted into a target tissue site (e.g., bone defect, void, fracture, etc.), the matrix will stay together at the target tissue site. In the context of putties containing calcium phosphate particles and insoluble collagen fibers, upon stretching, the putties can exhibit elongation, during which the existence of substantial levels of intermeshed collagen fibers clinging to one another becomes apparent.

In some embodiments, the matrix comprises no soluble collagen fibers. In some embodiments, the matrix comprises both soluble and insoluble collagen fibers.

In some embodiments, the implantable matrix is a putty comprising ceramic and collagen and the ceramic has a density of about 0.15 g/cc to about 0.45 g/cc and the collagen has a density of about 0.02 g/cc to about 1.0 g/cc of the putty and the putty comprises from about 60% to about 90% by volume of a liquid or about 60% to about 90% liquid volume percentage.

In some embodiments, the implantable matrix is a putty comprising ceramic and collagen and the ceramic has a density of about 0.10 g/cc and the collagen has a density of about 0.02 g/cc of the putty before the putty is hydrated with a liquid.

In some embodiments, the implantable matrix is a putty comprising ceramic and collagen and the ceramic has a density of about 0.29 g/cc and the collagen has a density of about 0.06 g/cc of the putty and the putty comprises a liquid that occupies from about 80% to about 85% by volume of the final volume of the putty after the putty is hydrated with a liquid.

As used herein, the term “shape-retaining” includes that the matrix (e.g.,, putty, flowable material, paste, etc.) is highly viscous and unless acted upon with pressure tends to remain in the shape in which it is placed. The pressure can be by hand, machine, or from the delivery device (injection from a syringe). In some embodiments, the shape retaining feature of the matrix can be contrasted to thinner liquid matrices or liquid paste forms, which readily flow, and thus would pool or puddle upon application to a surface.

In some embodiments, a combination of ingredients provide a medical putty material that not only, contains a significant, high level of large particulate mineral particles (e.g., calcium phosphate particles), but also exhibits superior properties with respect to malleability, cohesiveness, and shape retention.

In some embodiments, the matrix of the present application will include a combination of soluble collagen and insoluble collagen. “Soluble collagen”refers to the solubility of individual tropocollagen molecules in acidic aqueous environments. Tropocollagen may be considered the monomeric unit of collagen fibers and its triple helix structure is well recognized. “Insoluble collagen” as used herein refers to collagen that cannot be dissolved in an aqueous alkaline or in any inorganic salt solution without chemical modification, and includes for example hides, splits and other mammalian or reptilian coverings. For example, “natural insoluble collagen” can be derived from the corium, which is the intermediate layer of an annual hide e.g. bovine, porcine. etc.) that is situated between the grain and the flesh “Reconstituted collagen” is essentially collagen fiber segments that have been depolymerized into individual triple helical molecules, then exposed to solution and then reassembled into fibril-like forms.

In some embodiments, the matrix that is in the form of a putty contains both soluble collagen and insoluble collagen fibers, as well as the calcium phosphate particles that contain the therapeutic agent(s). The soluble collagen and insoluble collagen fibers can first be prepared separately, and then combined with the calcium phosphate particles. Both the soluble collagen and the insoluble collagen fibers can be derived from bovine hides, but can also be prepared from other collagen sources (e.g. bovine tendon, porcine tissues, recombinant DNA techniques, fermentation, etc.).

In certain embodiments the putty comprises insoluble collagen fibers at a level of 0.04 g/cc to 0.1 g/cc of the putty, and soluble collagen at a level of 0.01 g/cc to 0.08 g/cc of the putty. In other embodiments, the putty includes insoluble collagen fibers at a level of about 0.05 to 0.08 g/cc the putty, and soluble collagen at a level of about 0.02 to about 0.05 g/cc in the putty. In general, putties may include insoluble collagen fibers in an amount (percent by weight) that is at least equal to or greater than the amount of soluble collagen, to contribute beneficially to the desired handling and implant properties of the putty material. In some embodiments, when the putty contains collagen, the insoluble collagen fibers and soluble collagen can be present in a weight ratio of 4:1 to 1:1, more advantageously about 75:25 to about 60:40. Further still, additional desired putties include the insoluble collagen fibers and soluble collagen in a weight ratio of about 75:25 to about 65:35, and in one specific embodiment about 70:30. The insoluble collagen fibers, in some embodiments, will be in the composition more than the soluble collagen fibers.

One suitable putty for use in the present application is MasterGraft® Putty produced by Medtronic Sofamor Danek, Inc.

In some embodiments, when the matrix is a putty and comprises small ceramic particles, it is sufficiently flowable so that it can be pushed through a large or small needle/cannula.

In some embodiments, the matrix is in the form of medical putty that also includes an amount of a particulate mineral material (e.g., calcium phosphate). In certain embodiments, the particulate mineral is incorporated in the putty at a level of at least about 0.25 g/cc of putty, typically in the range of about 0.25 g/cc to about 0.35 g/cc. Such relatively high levels of mineral can be helpful in providing a scaffold for the ingrowth of new bone. The mineral component can also contain the therapeutic agent(s).

In some embodiments, the putty comprises a natural or synthetic mineral that is effective to provide a scaffold for bone ingrowth. Illustratively, the mineral comprises one or more materials comprising bone particles, Bioglass, tricalcium phosphate, biphasic calcium phosphate, hydroxyapatite, corraline hydroxyapatite, and biocompatible ceramics. Biphasic calcium phosphate is a particularly desirable synthetic ceramic for use in the present application. Such biphasic calcium phosphate can have a tricalcium phosphate: hydroxyapatite weight ratio of about 50:50 to about 95:5, more preferably about 85:15. The mineral material can be particulate having an average particle diameter between about 0.4 and 5.0 mm, more typically between about 0.4 and 3.0 mm, and desirably between about 0.4 and 2.0 mm.

A putty of the present application can include a significant proportion of a liquid carrier, which will generally be an aqueous liquid such as water, saline, dextrose, buffered solutions or the like. In one aspect, a malleable, cohesive, shape-retaining putty is provided that comprises about 60% to 75% by weight of an aqueous liquid medium, such as water, advantageously about 65% to 75 by weight of an aqueous liquid medium (e.g. water), based on the weight of the matrix (including therapeutic agents) and/or bioactive agent(s)) and the aqueous liquid medium.

In some embodiments the implantable matrix can be provided in any suitable shape including cylinders, cubes, irregular or other shapes. In one embodiment, the implantable matrix has one or more reservoirs defined therein for receiving amounts of a wetting liquid, e.g. to be used in the preparation of a wetted implant body or a wetted malleable formulation such as a putty, paste, or more flowable substance from the matrix.

In embodiments, the implantable matrix can be prepared using any suitable technique, including for example by casting a liquid medium into which the dry ingredients have been added, and then drying that medium by any appropriate means such as air drying or lyophilization. In many instances, such cast, dried bodies form surface features that tend to initially resist the absorption of liquids such as aqueous mediums. Other known preparative techniques such as molding, extrusion, machining, and the like, can also be used in the preparation of the matrix material.

### Minerals

The implantable matrix can also include a mineral component. The mineral used can include a natural or synthetic mineral that is effective to provide a scaffold for bone ingrowth. Illustratively, the mineral matrix may comprise one or more bone particles, Bioglass®, tricalcium phosphate, biphasic calcium phosphate, hydroxyapatite, corraline hydroxyapatite, and biocompatible ceramics. Biphasic calcium phosphate is a particularly desirable synthetic ceramic for use in the matrix. Such biphasic calcium phosphate can have a tricalcium phosphate:hydroxyapatite weight ratio of about 50:50 to about 95:5, more preferably about 70:30 to about 95:5, even more preferably about 80:20 to about 90:10, and most preferably about 85:15. The mineral material can be particulate having an average particle diameter between about 0.4 and 5.0 mm, more typically between about 0.4 and 3.0 mm, and desirably between about 0.4 and 2.0 mm.

In one aspect, the mineral material can include bone particles, possibly cancellous but preferably cortical, ground to provide an average particle diameter among those discussed above for the particulate mineral material. Both human and non-human sources of bone are suitable for use in the instant matrix, and the bone may be autographic, allographic or xenographic in nature relative to the mammal to receive the implant. Appropriate pre-treatments known in the art may be used to minimize the risks of disease transmission and/or immunogenic reaction when using bone particles as or in the mineral material.

In one embodiment, xenogenic bone that has been pretreated to reduce or remove its immunogenicity is used in or as the phorous mineral matrix in the implant composition. For example, the bone can be calcined or deproteinized to reduce the risks of immunogenic reactions to the implant material.

In some embodiments, the matrix may comprise mineral particles that offer compression resistance. In some embodiments, the particles comprise at least 10%, 15%, 20%, 25%, 30%, 35%, 40%, 45%, 50%, 60%, 65%, 70%, 80%, 85%, 90% or 95% by weight of the matrix. In some embodiments, the particles are predominantly any shape (e.g., round, spherical, elongated (powders, chips, fibers, cylinders, etc.).

In embodiments, the microporosity of the particles comprises from 5% to 50%, and in some embodiments, the microporosity of the particles comprises at 5% to 35% or at least 20%. In some embodiments, the microporosity of the particles comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50%.

In some embodiments, the particles are not entangled with each other but contact each other and portions of each particle overlap in the matrix to provide compression resistance. In some embodiments, at least 50%, 60%, 65%, 70%, 75%, 80%, 85%, 90%, 95%, or more of the particles overlap each other in the matrix.

In some embodiments, the particles are randomly distributed throughout the matrix. In other embodiments, the particles are uniformly or evenly distributed throughout the matrix. In some embodiments, the particles may be dispersed in the matrix using a dispersing agent. In other embodiments, the particles may be stirred in a polymer and the mechanical agitation will distribute the particles in the matrix until the desired distribution is reached (e.g., random or uniform).

In some embodiments, the matrix may comprise a resorbable ceramic (e.g., hydroxyapatite, tricalcium phosphate, bioglasses, calcium sulfate, etc.) tyrosine-derived polycarbonate poly (DTE-co-DT carbonate), in which the pendant group via the tyrosine—an amino acid—is either an ethyl ester (DTE) or free carboxylate (DT) or combinations thereof.

In some embodiments, the matrix may be seeded with harvested bone cells and/or bone tissue, such as for example, cortical bone, autogenous bone, allogenic bones and/or xenogenic bone. In some embodiments, the matrix may be seeded with harvested cartilage cells and/or cartilage tissue (e.g., autogenous, allogenic, and/or xenogenic cartilage tissue). For example, before insertion into the target tissue site, the matrix can be wetted with the graft bone tissue/cells, for example with bone tissue/cells aspirated from the patient, at a ratio of about 3:1, 2:1, 1:1, 1:3 or 1:2 by volume. The bone tissue/cells are permitted to soak into the matrix, and the matrix may be kneaded by hand or machine, thereby obtaining a pliable and cohesive consistency that may subsequently be packed into a bone defect. In some embodiments, the matrix provides a malleable, non-water soluble carrier that permits accurate placement and retention at the implantation site. In some embodiments, the harvested bone and/or cartilage cells can be mixed with the therapeutic agent(s) and/or other bioactive agent(s), and seeded in the interior of the matrix.

In some embodiments, the particles in the matrix comprise a resorbable ceramic, bone, synthetic degradable polymer, hyaluronic acid, chitosan or combinations thereof. In some embodiments, the particles comprise cortical, cancellous, and/or corticocancellous, allogenic, xenogenic or transgenic bone tissue. The bone component can be fully mineralized or partially or fully demineralized or combinations thereof. The bone component can comprise or consist of fully mineralized or partially or fully demineralized bone.

In some embodiments, the matrix comprises ceramic and/or collagen and the matrix can be impregnated with hyaluronic acid or collagen gel or gelatin (or other similar compounds) to both make the matrix more flowable in the needle/cannula and also prevent local muscle irritation by the particular therapeutic agent(s) and/or other bioactive agent(s).

In some embodiments, when the matrix comprises ceramic, the ceramic makes the matrix radiopaque so it can be seen on X-ray during injection to make sure it goes into the fracture site.

in some embodiments, the matrix may contain an inorganic material, such as an inorganic ceramic and/or bone substitute material. Examples of inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentic, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, by including inorganic ceramics, such as for example, calcium phosphate, in the matrix, this can facilitate the prevention of local bone resorption by providing slower release of a bioactive agent, e.g., a growth factor, due to its increased binding potential and also act as a local source of calcium and phosphate to the cells attempting to deposit new bone. The inorganic ceramic can also provide compression resistance and load bearing characteristics to the matrix.

In some embodiments, the matrix: (i) consists of only calcium phosphate particles containing the therapeutic agent(s); or (ii) consists essentially of the calcium phosphate particles containing the therapeutic agent(s); or (iii) comprises the calcium phosphate particles containing the therapeutic agent(s) and one or more other ingredients, surfactants, excipients or other ingredients or combinations thereof.

In some embodiments, the mineral particles in the matrix comprise tricalcium phosphate and hydroxyapatite in a about 80:20 to about 90:10. In some embodiments, the mineral particles in the matrix comprise tricalcium phosphate and hydroxyapatite in a ratio of about 85:15.

In some embodiments, the matrix comprises biphasic calcium phosphate having a tricalcium phosphate: hydroxyapatite weight ratio from about 50:50 to about 95:5. More preferable about 70:30 to about 95:5, even more preferably about 80:20 to about 90:10, and most preferable about 85:15. In one embodiment, the calcium phosphate has an approximate porosity of at least 20%. Generally the amount of calcium phosphate in the biomedical implant can be sufficient to allow for the formation of an osteoid in the bone void or target site. Further, the matrix must be such that the scaffold is maintained for a sufficient amount of time for osteoid formation and eventual bone formation.

In Some embodiments, the calcium phosphate particles can have an average particle size of at least 0.1 mm, but more preferably about 0.2 mm to about 2 mm, and most preferably about 0.2 mm to about 1.0 mm.

In some embodiments, the matrix has a density of between about 1.6 g/cm³, and about 0.05 g/cm³. In some embodiments, the matrix has a density of between about 1.1 g/cm³, and about 0.07 g/cm³. For example, the density may be less than about 1 g/cm³, less than about 0.7 g/cm³, less than about 0.6 g/cm³, less than about 0.5 g/cm³, less than about 0.4 g/cm³, less than about 0.3 g/cm³, less than about 0.2 g/cm³or less than about 0.1 g/cm³.

In some embodiments, the diameter or diagonal of the matrix can range from 1 mm to 50 mm. In some embodiments, the diameter or diagonal of the matrix can range from 1 mm to 30 mm, or 5 mm to 10 mm which is small enough to fit through an endoscopic cannula, but large enough to minimize the number of matrices needed to fill a large bone defect (e.g., osteochondral detect. In some embodiments, at the time of surgery, the matrix can be soaked with a bioactive agent, e.g., growth factor or statin and molded or cut by the surgeon to the desired shape to fit the tissue or bone defect.

In some embodiments, the matrix comprises biodegradable polymeric and/or non-polymeric material coated on a plurality of calcium phosphate particles or alternatively the plurality of calcium phosphate particles can be disposed in the matrix. In some embodiments, the coating on the particles is from about 0.01 mm to about 0.1 mm thick.

In some embodiments, tissue will infiltrate the matrix to a degree of about at least 50 percent within about 1 month to about 6 months after implantation of the matrix. In some embodiments, about 75 percent of the matrix will be infiltrated by tissue within about 2-3 months after implantation of the matrix. In some embodiments, the matrix will be substantially, e.g., about 90 percent or more, submerged in or enveloped by tissue within about 6 months after implantation of the matrix. In some embodiments, the matrix will be completely submerged in or enveloped by tissue within about 9-12 months after implantation.

In some embodiments, the matrix has a thickness of from 1 mm to 15 mm, or from about 2 mm to about 10 mm, or 3 mm to about 5 mm. Clearly, different bone defects (e.g., osteochondral defects) may require different thicknesses for the matrices.

### Method of Making Matrix

In some embodiments, the matrix may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

One form of manufacturing the matrix involves casting the matrix material in a mold. The matrix material can take on the shape of the mold such as, crescent, quadrilateral, rectangular, cylindrical, plug, or any other shape. Additionally, the surface of the mold may be smooth or may include raised features or indentations to impart features to the matrix. Features from the mold can be imparted to the matrix as the matrix material in the mold is dried. In particular aspects, a roughened or friction engaging surface can be formed on the superior surface and/or the inferior surface of the matrix body. In some embodiments, protuberances or raised portions can be imparted on the superior surface and/or the inferior surface from the mold. Such examples of protuberances or raised portions are ridges, serrations, pyramids, and teeth, or the like.

In some embodiments, in manufacturing the matrix, a mixture of the matrix material (e.g., collagen) is combined with the elongated particles and a liquid to wet the material and form a slurry. Any suitable liquid can be used including, for example, aqueous preparations such as water, saline solution (e.g. physiological saline), sugar solutions, protic organic solvents, or liquid polyhydroxy compounds such as glycerol and glycerol esters, or mixtures thereof. The liquid may, for example, constitute about 5 to about 70 weight percent of the mixed composition prior to the molding operation. Certain liquids such as water can be removed in part or essentially completely from the formed matrix using conventional drying techniques such as air drying, heated drying, lyophilization, or the like.

In one embodiment of manufacture, a collagen mixture can be combined with particles, e.g., mineral particles, and a liquid, desirably with an aqueous preparation, to form a slurry. Excess liquid can be removed from the slurry by any suitable means, including tier example by applying the slurry to a liquid-permeable mold or form and draining away excess liquid.

In embodiments where the matrix includes collagen containing materials, before, during or after molding, including in some instances the application of compressive force to the collagen containing material, the collagen material can be subjected to one or more additional operations such as heating, lyophilizing and/or crosslinking to make the porous collagen interior or exterior of the matrix the desired porosity. in this regard, crosslinking can be used to improve the strength of the formed matrix. Alternatively, one or more of the surface of the matrix can be crosslinked to reduce the size of the pores of the porous interior and thereby form the exterior of the matrix that is less permeable and/or less porous than the porous interior. Crosslinking can be achieved, for example, by chemical reaction, the application of energy such as radiant energy (e.g. UV light or microwave energy), drying and/or heating and dye-mediated photo-oxidation; dehydrothermal treatment; enzymatic treatment or others.

In some embodiments, chemical crosslinking agents are used, including those that contain bifunctional or multifunctional reactive groups, and which react with matrix. Chemical crosslinking can be introduced by exposing the matrix material to a chemical crosslinking agent, either by contacting it with a solution of the chemical crosslinking agent or by exposure to the vapors of the chemical crosslinking agent. This contacting or exposure can occur before, during or after a molding operation. In any event, the resulting material can then be washed to remove substantially all remaining amounts of the chemical crosslinker if needed or desired for the performance or acceptability of the final implantable matrix.

Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as other heterobifunctional crosslinking agents; hexamethylene diisocyante; and/or sugars, including glucose, can also crosslinking the matrix material.

In some embodiments, the matrices are formed by mixing particles, e.g., mineral particles, in with a polymer slurry such as collagen and pouring into a shaped mold. The composite mixture is freeze dried and possibly chemically crosslinked and cut to the final desired shape.

In some embodiments, the matrix may comprise sterile and/or preservative free material, The matrix can be implanted by hand or machine in procedures such as for example, laparoscopic, arthroscopic, neuroendoscopic, endoscopic, rectoscopic procedures or the like.

The matrix of the present application may be used to repair bone and/or cartilage at a target tissue site, e.g., one resulting from injury, defect brought about during the course of surgery, infection, malignancy or developmental malformation. The matrix can be utilized in a wide variety of orthopedic, periodontal, neurosurgical, oral and maxillofacial surgical procedures such as the repair of simple and/or compound fractures and/or non-unions; external and/or internal fixations; joint reconstructions such as arthrodesis; general arthroplasty; cup arthroplasty of the hip; femoral and humeral head replacement; femoral head surface replacement and/or total joint replacement; repairs of the vertebral column including spinal fusion and internal fixation; tumor surgery, e.g., deficit filling; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay implantable matrices; implant placement and revision; sinus lifts; cosmetic procedures; etc. Specific bones which can be repaired or replaced with the implantable matrix herein include the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal and/or metatarsal bones.

### Bionetive Agents

In some embodiments, the matrix further comprises bioactive agents in addition to the therapeutic agent(s) described above. The term “bioactive agent”, as used herein, is used in its broadest sense and includes any substance or mixture of substances that have clinical use. Consequently, bioactive agents may or may not have pharmacological activity per se, e.g., a dye, or fragrance. Alternatively a bioactive agent could be any agent that provides a therapeutic or prophylactic effect, a compound that affects or participates in tissue growth, cell growth, cell differentiation, an anti-adhesive compound, a compound that may be able to invoke a biological action such as an immune response, or could play any other role in one or more biological processes.

Examples of classes of bioactive agents which may be utilized in accordance with the present disclosure include anti-adhesives, analgesics, antipyretics, anesthetics, antieileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, steroids, lipids, lipids, lipopolysaccharides, platelet activating drugs, clotting factors and enzymes. It is also intended that combinations of bioactive agents may be used.

Bioactive agents may also be provided by tissue materials, including for instance autologous tissue materials, which are incorporated into the material to be implanted in the patient. Such tissue materials can include blood or blood fractions, bone or bone marrow fractions, and/or other sources of cells or other beneficial tissue components derived. from the patient to be treated or another suitable animal source.

Bioactive agents such as those described herein can be incorporated homogeneously or regionally into an implantable material by simple admixture or (otherwise, and/or may be incorporated into a three-dimensional implant body and/or a final wetted (preferably conformable) medical material in conjunction with another carrier form or medium such as microspheres or another microparticulate formulation. Suitable techniques for forming microparticles are well known in the art, and can be used to entrain or encapsulate bioactive agents, whereafter the microparticles can be dispersed within the implantable material upon forming the three-dimensional body and/or upon wetting the body (e.g. by incorporating the microparticles in the wetting liquid).

### Growth Factors

In some embodiments, a growth factor and/or the therapeutic agent(s) may be disposed on or in the matrix by hand, electrospraying, ionization spraying or impregnating, vibratory dispersion (including sonication), nozzle spraying, compressed-air-assisted spraying, injecting, brushing and/or pouring. For example, a growth factor such as rhBMP-2 may be disposed on or in the matrix by the surgeon before the matrix is administered or the matrix may be pre-loaded with the growth factor by the manufacturer beforehand.

The implantable matrix may comprise at least one growth factor. These growth factors include osteoinductive agents (e.g., agents that cause new bone growth in an area where there was none) and/or osteoconductive agents (e,g., agents that cause ingrowth of cells into and/or through the matrix). Osteoinductive agents can be polypeptides or polynucleotides compositions. Polynucleotide compositions of the osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Such gene therapy and delivery techniques are known in the art (see, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In some embodiments, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin, precipitating agents or the like. Optionally, gene therapy compositions can be delivered in Liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in U.S. Pat. No. 5,652,224, which is herein incorporated by reference.

Polypeptide compositions of the isolated osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides. Polypeptide compositions of the osteoinductive agents include, but are not limited to, full length proteins, fragments or variants thereof.

Variants of the isolated osteoinductive agents include, but are not limited to, polypeptide variants that are designed to increase the duration of activity of the osteoinductive agent in vivo. Typically, variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides). In some embodiments, the isolated osteoinductive agent(s) are provided as fusion proteins. In one embodiment, the osteoinductive agent(s) are available as fusion proteins with the Fc portion of human IgG. In another embodiment, the osteoinductive agent(s) are available as hetero- or homodimers or multimers. Examples of some fusion proteins include, but are not limited to, ligand fusions between mature osteoinductive polypeptides and the Fc portion of human Immunoglobulin G (IgG). Methods of making fusion proteins and constructs encoding the same are well known in the art.

Isolated osteoinductive agents that are included within the matrix are typically sterile. In a non-limiting method, sterility is readily accomplished for example by filtration through sterile filtration membranes (e.g., 0.2 micron membranes or filters). In one embodiment, the matrix includes osteoinductive agents comprising one or more members of the family of Bone Morphogenic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2,BMP-3, BMP-5, BMP-6, BMP-7, BMP-8, BMP-9, BMP-10, BMP-11, BMP-12, BMP-13, BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BNIP-7; BMP-8; BMP-9; P-1.0; BMP-11; BMP-12; BMP-13; BMP-15; BMP-16; BMP-17; or BMP-18; as well as any combination of one or more of these BMPs including full length BMPs or fragments thereof or combinations thereof; either as polypeptides or polynucleotides encoding the polypeptide fragments of all of the recited BMPs. The isolated BMP osteoinductive agents may be administered as polynucleotides, polypeptides, full length protein or combinations thereof.

In another embodiment, isolated osteoinductive agents that are loaded in the matrix include osteoclastogenesis inhibitors to inhibit bone resorption of the bone tissue surrounding the site of implantation by osteoclasts. Osteoclast and osteociastogenesis inhibitors include, but are not limited to, osteoprotegerin polynucleotides or polypeptides, as well as mature osteoprotegerin proteins, polypeptides or polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors that can be loaded in the matrix further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entireties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entirety), or acylguanidine compounds such as those described in U.S. Pat. No. 6,492,356 (herein incorporated by reference in its entirety).

In another embodiment, isolated osteoinductive agents that can be loaded in the matrix include one or more members of the family of Connective Tissue Growth Factors (“CTGFs”). CTGE's are a class of proteins thought to have growth-promoting activities on connective tissues. Known members of the CTGF family include, but are not limited to, CTGF-1, CTGF-2, CTGF-4 polynucleotides or polypeptides thereof, as well as mature proteins, polypeptides or polynucleotides encoding the same.

In another embodiment, isolated osteoinductive agents that can be loaded in the matrix include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D, VEGF-E or polynucleotides or polypeptides thereof, as well as mature VEGF-A, proteins, polypeptides or polynucleotides encoding the same.

In another embodiment, isolated osteoinductive agents that can be loaded in the matrix include one or more members of the family of Transforming Growth Factor-beta (“TGFbetas”). TGF-betas are a class of proteins thought to have growth-promoting activities on a range of tissues, including connective tissues. Known members of the TGF-beta family include, but are not limited to, TGF-beta-1, TGF-beta-2, TGF-beta-3, polynucleotides or potypeptides thereof, as well as mature protein, polypeptides or polynucleotides encoding the same.

In another embodiment, isolated osteoinductive agents that can be loaded in the matrix include one or more Growth Differentiation Factors (“GDFs”). Known GDFs include, but are not limited to, GDF-1, GDF-2, GDF-3, GDF-7, GDF-10, GDF-11, and GDF-15. For example, GDFs useful as isolated osteoinductive agents include, but are not limited to, the following GMFs: GDF-1 polynucleotides or polypeptides corresponding to GenBank Accession Numbers M62302, AAA58501, and AAB94786, as well as mature GDF-1 polypeptides or polynucleotides encoding the same. GDF-2 polynucleotides or polypeptides corresponding to GenBank Accession Numbers BC069643, BC074921, Q9UK05, AAH69643, or AAH74921, as well as mature GDF-2 polypeptides or polynucleotides encoding the same. GDF-3 polynucleotides or polypeptides corresponding to GenBank Accession Numbers AF263538, BC030959, AAF91389, AAQ89234, or Q9NR23, as well as mature GDF-3 polypeptides or polynucleotides encoding the same. GDF-7 polynucleotides or polypeptides corresponding to GenBank Accession Numbers AB158468, AF522369, AAP97720, or Q7Z4P5, as well as mature GDF-7 polypeptides or polynucleotides encoding the same. GDF-10 polynucleotides or polypeptides corresponding to GenBank Accession Numbers BC028237 or AAH28237, as well as mature GDF-10 polypeptides or polynucleatides encoding the same.

GDF-11 polynucleotides or polypeptides corresponding to CienBank Accession Numbers AF100907, NP_005802 or 095390, as well as mature GDF-11 potypeptides or polynucleotides encoding the same. GDF-15 polynucleotides or polypeptides corresponding to GenBank Accession Numbers BC008962, BC000529, AAH00529, or NP_004855, as well as mature GDF-15 polypeptides or polynucleotides encoding the same.

In another embodiment, isolated osteoinductive agents that can be loaded in the matrix include Cartilage Derived Motphogenic Protein (CDMP) and Lim Mineralization Protein (LMP) polynucleotides or polypeptides. Known CDMPs and LMPs include, but are not limited to, CDMP-1, CDMP-2, LMP-1, LMP-2, or LMP-3.

CDMPs and LMPs useful as isolated osteoinductive agents that can be loaded in the matrix include, but are not limited to, the following CDMPs and LMPs: CDMP-1 polynucleotides and polypeptides corresponding to CienBank Accession Numbers NM_000557, U13660, NP_000548 or P43026, as well as mature CDMP-1 polypeptides or polynucleotides encoding the same. CDMP-2 polypeptides corresponding to GenBank Accession Numbers or P55106, as well as mature CDMP-2 polypeptides. LMP-1 polynucleotides or polypeptides corresponding to GenBank Accession Numbers AF345904 or AAK30567, as well as mature LMP-1 polypeptides or polynucleotides encoding the same. LMP-2 polynucteotides or polypeptides corresponding to GenBank Accession Numbers AF345905 or AAK30568, as well as mature LMP-2 polypeptides or polynucleotides encoding the same. LMP-3 polynucleotides or polypeptides corresponding to GenBank Accession Numbers AF345906 or AAK30569, well as mature LMP-3 polypeptides or polynucleotides encoding the same.

In another embodiment, isolated osteoinductive agents that can be loaded in the matrix include one or more members of any one of the families of Bone Morphogenic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), or Transforming Growth Factor-betas (TGF-betas), as well as mixtures or combinations thereof.

In another embodiment, the one or more isolated osteoinductive agents that can be loaded in the matrix are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, or any combination thereof; CTGF-1, CTGF-2, CGTF-3, CTGF-4, or any combination thereof; VEGF-A, VEGF-B, VEGF-C, VEGF-D, VEGF-E, or any combination thereof; GDF-1, GDF-2, GDF-3, GDF-7, GDF-10, GDF-11, GDF-15, or any combination thereof; CDMP-1, CDMP-2, LMP-1, LMP-2, LMP-3, and/or any combination thereof; Osteoprotegerin; TGF-beta-1, TGF-beta-2, TGF-beta-3, or any combination thereof; or any combination of one or more members of these groups.

In some embodiments, BMP-2, BMP-7 and/or GDF-5 may be used at 1-2 mg/cc of matrix. The concentrations of growth factor can be varied based on the desired length or degree of osteogenic effects desired. Similarly, one of skill in the art will understand that the duration of sustained release of the growth factor can be modified by the manipulation of the compositions of the matrix, such as for example, microencapsulation of the growth factor within polymers. The sustained release matrix can therefore be designed to provide customized time release of growth factors that stimulate the natural healing process.

The therapeutic agent(s) and/or bioactive agent(s), e.g., growth factor, may contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and alcohol; solubility adjusting agents; stabilizers; and/or cohesion modifiers. In some embodiments, the bioactive agent(s) may comprise sterile and/or preservative free material.

These above inactive ingredients may have multi-functional purposes including the carrying, stabilizing and controlling the release of the bioactive agent(s) (e.g., growth factor) and/or other therapeutic agent(s). The sustained release process, for example, may be by a solution-diffusion mechanism or it may be governed by an erosion-sustained process.

In some embodiments, an implantable matrix comprising a growth factor is provided, wherein the formulation is a freeze-dried or lyophilized formulation. Typically, in the freeze-dried or lyophilized formulation an effective amount of a growth factor is provided. Lyophilized formulations can be reconstituted into solutions, suspensions, emulsions, or any other suitable form for administration or use. The lyophilized formulation may comprise the liquid used to reconstitute the growth factor. Lyophilized formulations are typically first prepared as liquids, then frozen and lyophilized. The total liquid volume before lyophilization can be less, equal to, or more than the final reconstituted volume of the lyophilized formulation. The lyophilization process is well known to those of ordinary skill in the art, and typically includes sublimation of water from a frozen formulation under controlled conditions.

Lyophilized formulations can be stored at a wide range of temperatures. Lyophilized formulations may be stored at or below 30° C., for example, refrigerated at 4° C., or at room temperature (e.g., approximately 25° C.).

Lyophilized formulations of the growth factor are typically reconstituted for use by addition of an aqueous solution to dissolve the lyophilized formulation. A wide variety of aqueous solutions can be used to reconstitute a lyophilized formulation. In some embodiments, lyophilized formulations can be reconstituted with a solution containing water (e.g., USP WFI, or water for injection) or bacteriostatic water (e.g., USP WFI with 0.9% benzyl alcohol). However, solutions comprising buffers and/or excipients and/or one or more pharmaceutically acceptable carries can also be used. In some embodiments, the solutions do not contain any preservatives (e.g., are preservative free).

### Other Bioactive Agents

Examples of other bioactive agents include but are not limited to IL-1 inhibitors, such Kineret® (anakinra), which is a recombinant, non-glycosylated form of the human interleukin-1 receptor antagonist (IL-1Ra), or AMG 108, which is a monoclonal antibody that blocks the action of IL-1. Bioactive agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), and aurin-tricarboxylic acid (which inhibits TNF-α), may also be useful as bioactive agents for reducing inflammation. It is further contemplated that where desirable a pegylated form of the above may be used. Examples of stilt other bioactive agents include NF kappa B inhibitors such as antioxidants, such as dithiocarbamate, and other compounds, such as, for example, sulfasalazine.

In embodiments, the implantable matrix comprises one or more bioactive agents chosen from an anti-inflammatory agent, analgesic agent, an osteoinductive growth factor or a combination thereof. Examples of anti-inflammatory agents include, but are not limited to, apazone, celecoxib, dictofenac, diflunisal, enolic acids (piroxicam, meloxicam), etodolac, fenamates (mefenamic acid, meclofenamic acid), gold, ibuprofen, indomethacin, ketoprofen, ketorolac, naburnetone, naproxen, nimesulide, salicylates, sulfasalazine [2-hydroxy-5-[-4-[C2-pyridinylamino)sulfonyl]azo]benzoic acid, sulindac, tepoxalin, and tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Examples of analgesic agents include, but are not limited to, acetaminophen, bupivicaine, fluocinolone, lidocaine, opioid analgesics such as buprenorphine, butorphanol, dextromoramide, dezocine, dextropropoxyphene, diamorphine, fentanyl, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

In some embodiments, the matrix can comprise a statin. Examples of statins include, but are not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein. incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

In some embodiments, one or more bioactive agents (including one or more growth factors) may be disposed on or in the interior of the matrix by hand, electrospraying, ionization spraying or impregnating, vibratory dispersion (including sonication), nozzle spraying, compressed-air-assisted spraying, injecting, brushing and/or pouring.

Application of the bioactive agent, e.g., growth factor, to the matrix may occur at the time of surgery or by the manufacturer or in any other suitable manner. For example, bioactive agent, e.g., a growth factor, may be further reconstituted using a syringe and the syringe can be placed into the interior of the matrix via insertion of a needle or cannula (piercing the matrix) and placing it into the interior of the matrix and injecting the agent so it is evenly distributed throughout the porous interior.

In some embodiments, the agent may be applied to the matrix (i.e., collagen) prior to combining the materials and forming it into the final matrix shape. Indeed, the agent can be blended into the natural or synthetic polymer (i.e., POE) and poured into molds of the final shape of the matrix. Alternatively, the agent, such as a growth factor, e.g., bone morphogenetic protein, in a suitable liquid carrier, may be applied onto and/or into the porous loaded matrix after forming it into the final shape by soaking, dripping, injecting, spraying, etc.

### Kits

The matrix, therapeutic agent(s), other bioactive agent(s) and devices to administer the implantable matrix composition may be sterilizable. In various embodiments, one or more components of the matrix, and/or medical device to administer it may be sterilizable, e.g., by radiation, in a terminal sterilization step in the final packaging. Terminal sterilization of a product can provide greater assurance of sterility than from processes such as an aseptic process, which require individual product components to be sterilized separately and the final package assembled in a sterile environment.

In various embodiments, a kit is provided comprising the therapeutic agent(s), bioactive agent(s), matrix, and/or diluents. The kit may include additional parts along with the implantable matrix combined together to be used to implant the matrix (e.g., wipes, needles, syringes, etc.). The kit may include the matrix in a first compartment. The second compartment may include a vial holding the therapeutic and/or bioactive agent(s), diluent and any other instruments needed for the localized drug delivery. A third compartment may include gloves, drapes, wound dressings and other procedural supplies for maintaining sterility of the implanting process, as well as an instruction booklet, which may include a chart that shows how to implant the matrix. A fourth compartment may include additional needles and/or sutures. Each tool may be separately packaged in a plastic pouch that is radiation sterilized. A fifth compartment may include an agent for radiographic imaging. A cover of the kit may include illustrations of the implanting procedure and a clear plastic cover may be placed over the compartments to maintain sterility.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is

1. An implantable matrix comprising an effective amount of at least one therapeutic agent having hemostatic and antimicrobial activity disposed in a biodegradable polymer, wherein the implantable matrix is configured to be implanted into a bone defect and release the therapeutic agent.

2. An implantable matrix according to claim 1, wherein (i) the implantable matrix is moldable and comprises at least one therapeutic agent comprising a hemostatic agent and an antimicrobial agent that are different agents; or (ii) the therapeutic agent comprises silver nitrate.

3. An implantable matrix according to claim 2, wherein the hemostatic agent is present in an amount of 0.001% to 10 wt % based on the total weight of the matrix.

4. An implantable matrix according to claim 3, wherein the hemostatic agent is present in an amount in the range from 0.1 to 5 wt % based on the total weight of the matrix. An implantable matrix according to claim 3, wherein the antimicrobial agent s present in an amount of from 0.1 wt % to 1 wt % based on the total weight of the matrix.

6. An implantable matrix according to claim 5, wherein the antimicrobial agent is present in an amount in the range from 0.01 to 0.5 wt % based on the total weight of the matrix.

7. An implantable matrix according to claim 2, wherein the hemostatic agent comprises silver nitrate, gelatin, collagen, oxidized cellulose, doxycycline, tetracycline, polidocanol, cyanoacrylate, thrombin, fibrin, chitosan, ascorbic acid, chitosan, ferric sulfate, fibrinogen. iron oxyacid, a sodium salt of N-acyl-5-bromo(3,5-dibromo) anthranilic acid, bleomycin, clarithromycin, erythromycin, sotradecol, ankaferd, rutin, or a combination thereof,

8. An implantable matrix according to claim 2, wherein the antimicrobial comprises an antibiotic, an antifungal, an antiviral or combinations thereof.

9. An implantable matrix according to claim 8, wherein the antimicrobial agent comprises a metal comprising silver, copper, platinum, gold or mixtures thereof.

10. An implantable matrix according to claim 1, wherein the at least one therapeutic agent comprises a multifunctional therapeutic agent having both hemostatic and antimicrobial activity.

11. An implantable matrix according to claim 10, wherein the multifunctional therapeutic agent is present in an amount up to 25 wt %, based on the total weight of the implantable matrix.

12. An implantable matrix according to claim 11, wherein the multifunctional therapeutic agent is present in an amount in the range from 5 to 20 wt %, based on the total weight of the matrix.

13. An implantable matrix according to claim 10, wherein the multifunctional therapeutic agent comprises silver nitrate particles having an average particle size greater than 1 micron, chitosan niacinamide ascorbate salt and combinations thereof

14. An implantable matrix according to claim 1, wherein the therapeutic agent comprises silver nitrate.

15. An implantable matrix according to claim 14, wherein the matrix further comprises a mineral.

16. An implantable matrix according to claim 15, wherein the matrix is in the form of a putty or paste.

17. An implantable matrix according to claim 15, wherein the multifunctional therapeutic agent is silver nitrate particles having an average particle size greater than 1 micron.

18. A method of treating a bone defect in which the bone defect site possesses at least one cavity, the method comprising inserting an implantable matrix, the implantable matrix comprising an effective amount of at least one therapeutic agent having hemostatic and antimicrobial activity disposed in a biodegradable polymer, wherein the implantable matrix allows influx of at least progenitor, bone and/or cartilage cells therein.

19. A method of treating a bone defect of claim 18, wherein the hemostatic agent is present in an amount from 0.1 to 5 wt % based on the total weight of the matrix.

20. A method of treating a bone defect of claim 18, wherein the therapeutic agent comprises silver nitrate.
